(19) United States
(12) Patent Application Publication
(10) Pub. No.: US 2025/0143686 A1
(43) Pub. Date: May 8, 2025

(54) SURGICAL ANCHORS AND SURGICAL ANCHORING METHODS

(71) Applicant: INTERNATIONAL LIFE SCIENCES, LLC d/b/a ARTELON, MARIETTA, GA (US)

(72) Inventors: Lauren Chase Thornburg, Cumming, GA (US); Aaron Christopher Smith, Marietta, GA (US); Mark Elliot Wiltshire, Fort Collins, CO (US)

(21) Appl. No.: 18/691,032

(22) PCT Filed: Jan. 11, 2023
(86) PCT No.: PCT/US2023/060462

§ 371 Date: Mar. 11, 2024

Related U.S. Application Data
(60) Provisional application No. 63/305,039, filed on Jan. 31, 2022.

(51) Int. Cl. A61B 17/04; A61B 17/00

(52) U.S. Cl. CPC A61B 17/0401; A61B 2017/00831

(57) ABSTRACT

Surgical anchors and related systems and methods that utilize sutures or other flexible, elongated members to actively deploy a deformable anchor body in a manner that facilitates better deployment of the anchor in the bone, resulting in better fixation and resistance to pull out.

### CROSS-REFERENCES TO RELATED APPLICATIONS

The present application claims priority to U.S. Provisional Application No. 63/305,039, entitled “SURGICAL ANCHORS AND SURGICAL ANCHORING SYSTEMS AND METHODS” filed Jan. 31, 2022, the entire contents of which are herein incorporated by reference in its entirety for all purposes.

### BACKGROUND OF THE INVENTION

There are several surgical anchors currently on the market that utilize one or more sutures in combination with a deformable anchor body. One example is the Biomet JuggerKnot. The Biomet Juggerknot is a passive deployment anchor. It does not include a mechanism for actively and intentionally deploying the anchor. Rather, the anchor is deployed by pulling the anchor body up through the bone cavity, such that resistance between the bone cavity and the anchor body results in the anchor body balling up or otherwise enlarging to a point where the anchor body resists further withdrawal from the cavity. This type of passive deployment may be undesirable, as there is the potential for the anchor to not fully deploy and be inadequately anchored. There remains room for improvement in the field of surgical anchors.

### BRIEF SUMMARY OF THE INVENTION

In this patent, we describe several examples of surgical anchors and related systems and methods that utilize sutures or other flexible, elongated members to actively deploy a deformable anchor body in a manner that facilitates better deployment of the anchor in the bone, resulting in better fixation and resistance to pull out. The surgical anchors disclosed in this patent provide an unexpectedly high resistance to pull out in a minimally invasive size.

In one example, a surgical anchor includes: (a) an anchor body, the anchor body deformable from an insertion configuration to an anchoring configuration; and (b) a flexible, elongated member, the flexible, elongated member including: (i) a looped section, the looped section including a proximal portion and a distal portion, wherein at least part of the distal portion is engaged with the deformable anchor body; and (ii) first and second tail sections, the first and second tail sections extending from a proximal portion of the looped section; in which the surgical anchor is configured such that tensioning the first and second tail sections causes the loop to narrow, causing the anchor body to deform to the anchoring configuration.

In some implementations, the first and second tail sections connect to the looped section at a proximal apex of the looped section.

In some implementations, the looped section is formed by two overlapping loops of the elongated member and the looped section ends where the first and second tail sections connect to the looped section.

In some implementations, the looped section includes more than one complete loop of the elongated member, with the elongated member, including the looped section and the two tail sections, being a single suture.

In some implementations, the surgical anchor is configured such that tensioning the first and second tail sections causes the loop to narrow and causes the connection between the looped section and the tail sections to move distally, causing the anchor body to deform to the anchoring configuration.

In some implementations, the first and second tail sections of the elongated member pierce the elongated member at the proximal portion of the looped section to connect the first and second tail sections to the looped section.

In some implementations, tensioning the first and second tail sections draws the tail sections further through the elongated member where the tail sections pierce the elongated member.

In some implementations, tensioning the first and second tail sections tightens the looped section.

In some implementations, the anchor body includes a central body portion and two wings.

In some implementations, tensioning the first and second tail sections causes the central body to expand in size and causes the two wings to press outwardly.

In some implementations, the anchor body is a sleeve.

In some implementations, the looped section of the elongate member extends through a cavity of the sleeve.

In some implementations, the looped section extends out of the sleeve adjacent to the two wings.

In some implementations, the looped section extends out of the sleeve adjacent to the two wings.

In another example, a surgical anchor includes: (a) an anchor body, the anchor body deformable from an insertion configuration to an anchoring configuration; and (b) a single length of suture, the single length of suture including: (i) a looped section, the looped section including more than one complete loops of the single length of suture; and (ii) first and second tail sections, the first and section tail sections extending from the looped section; the surgical anchor is configured such that tensioning the first and second tail sections causes the looped section to narrow, causing the anchor body to deform to the anchoring configuration.

In another example, a surgical anchoring method includes: (a) using an inserter, inserting a surgical anchor into a bone cavity, the surgical anchor including: (1) an anchor body, the anchor body deformable from an insertion configuration to an anchoring configuration; and (2) a flexible, elongated member, the flexible, elongated member including: (i) a looped section, the looped section including a proximal portion and a distal portion, in which at least part of the distal portion is engaged with the deformable anchor body; and (ii) first and second tail sections, the first and second tail sections connected to the looped section at the proximal portion of the looped section; and (b) tensioning the first and second tail sections to cause the connection between the tail sections and the looped section to move distally, causing the anchor body to deform to the anchoring configuration, anchoring the surgical anchor in the bone cavity.

In some implementations, the bone cavity extends through cortical bone and into cancellous bone, and inserting the surgical anchor includes inserting the surgical anchor into the cancellous bone such that the anchor body is spaced from and below the cortical bone.

In some implementations, the first and second tail sections are tensioned and the anchor body deformed to the anchoring configuration while the anchor body is spaced from and below the cortical bone.

In some implementations, the bone cavity is between about 1 mm and 2 mm in diameter, and, after anchoring the surgical anchor in the bone cavity, the surgical anchor is configured to resist a pullout force of at least 150 N.

In some implementations, the first and second tail sections are tensioned and the anchor body deformed to the anchoring configuration without drawing the anchor body proximally in the bone cavity.

In some implementations, the anchor body is on a distal end of the surgical instrument, and the distal end of the surgical instrument remains stationary during deformation of the anchor body.

In some implementations, the inserter includes an elongated shaft extending along an insertion axis, and, during tensioning of the first and second tail sections, the tail sections extend from the connection between the tail sections and the looped section at non-parallel angles relative to the insertion axis.

In some implementations, (i) the inserter includes an elongated shaft and a tensioner; (ii) the tensioner is moveable relative to the elongated shaft between an un-tensioned position and a tensioned position; (iii) the anchor body of the surgical anchor is on a distal end of the elongated shaft; and (iv) the first and second tail sections of the elongated member extend from the anchor body to the proximal tensioner; such that moving the tensioner from the un-tensioned position to the tensioned position causes the anchor body to deform to the anchoring configuration.

In some implementations, the inserter includes positive stops at the un-tensioned and tensioned positions.

### BRIEF DESCRIPTION OF THE DRAWINGS

FIG. 1a shows an example of a surgical anchor mounted on an inserter, with the anchor body in an insertion configuration.

FIG. 1b shows a close up of a surgical anchor mounted on a distal end of an inserter, with the anchor body in an insertion configuration.

FIG. 1c shows the surgical anchor and inserter of FIG. 1a, with the anchor body in an anchoring configuration.

FIG. 1d shows a close up of the surgical anchor and distal end of the inserter of FIG. 1c.

FIG. 2a shows an example of a surgical anchor.

FIG. 2b shows the surgical anchor of FIG. 2a, without the anchor body.

FIG. 2c shows a close up of the distal end of the surgical anchor of FIG. 2a.

FIG. 2d shows the close up of FIG. 2c, with the anchor body shown in phantom lines.

FIGS. 3a-b show an example of a surgical anchor mounted on an inserter, with the anchor body in an insertion configuration.

FIG. 3c show the surgical anchor and inserter of FIGS. 3a-b, with the anchor body deformed to an anchoring configuration.

FIGS. 4-10 shown an example of a surgical anchoring method.

FIG. 11a-c show an example of an inserter, with FIG. 11b showing a close up of the inserter's handle and FIG. 11c showing a close up of the inserter's tensioner.

FIGS. 12a-b show another example of an inserter in cross-section, with FIG. 12a showing the tensioner in a position that corresponds to an insertion (un-deployed) configuration of the surgical anchor and FIG. 12b showing the tensioner in a position that corresponds to an anchoring (deployed) configuration of the surgical anchor.

FIG. 13 shows an example of a distal end of an inserter.

FIGS. 14a-b show an example of an inserter in a guide, with FIG. 14a showing the inserter not fully seated in the guide and FIG. 14b showing the inserter fully seated in the guide.

FIGS. 15-18 illustrate testing of a surgical anchor in accordance with the present disclosure.

### DETAILED DESCRIPTION OF THE INVENTION

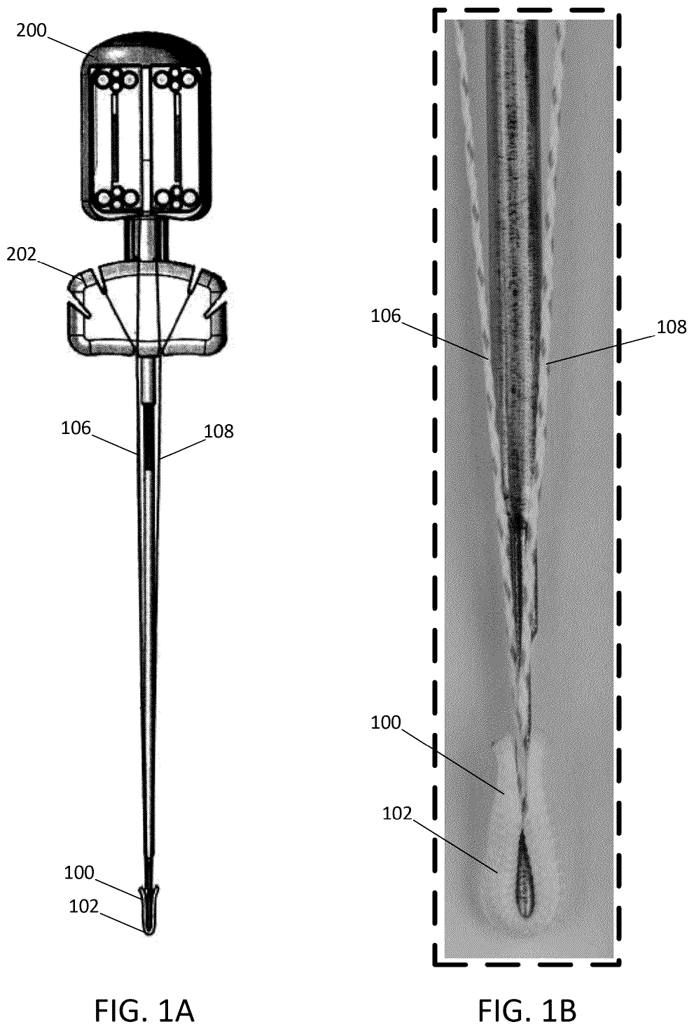

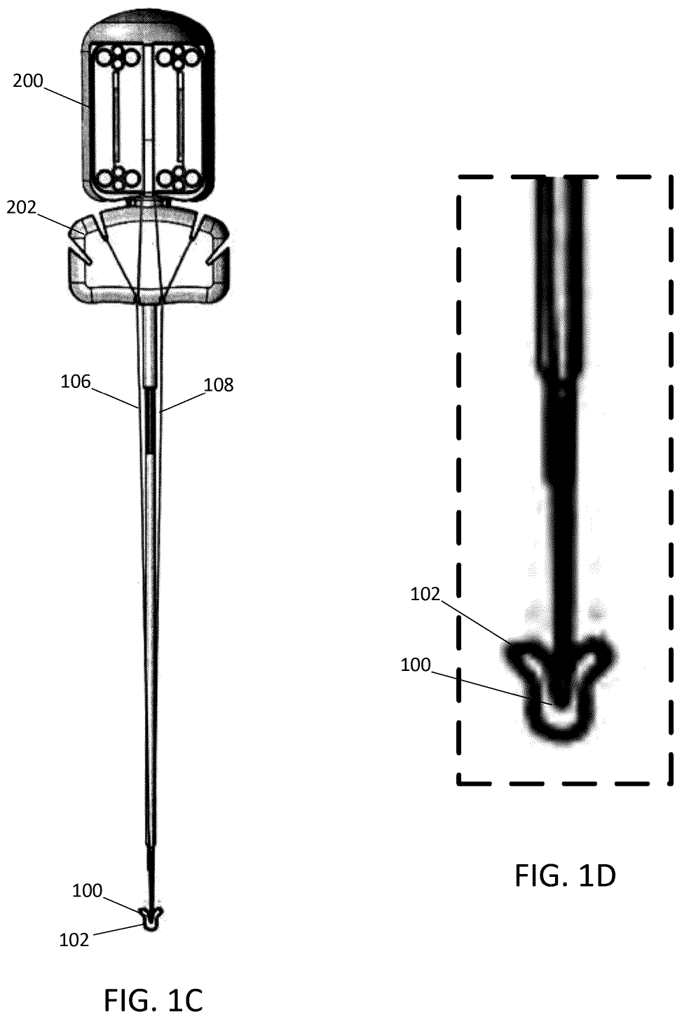

FIGS. 1a-d show an example of a surgical anchor 100 and an inserter 200. The anchor 100 includes an anchor body 102 positioned on a distal tip of the inserter 200, and a flexible, elongated member 104 including two tail sections 106, 108 extending from the anchor body 102 towards a proximal end of the inserter 200. The anchor body 102 is deformable from an insertion configuration (shown in FIGS. 1a-b) to an anchoring configuration (shown FIGS. 1c-d). The inserter 200 includes a tensioner 202 that can be actuated to tension the two tail sections 106, 108 of the surgical anchor 100, causing the anchor body 102 to deform from its insertion configuration to its anchoring configuration.

### Surgical Anchor

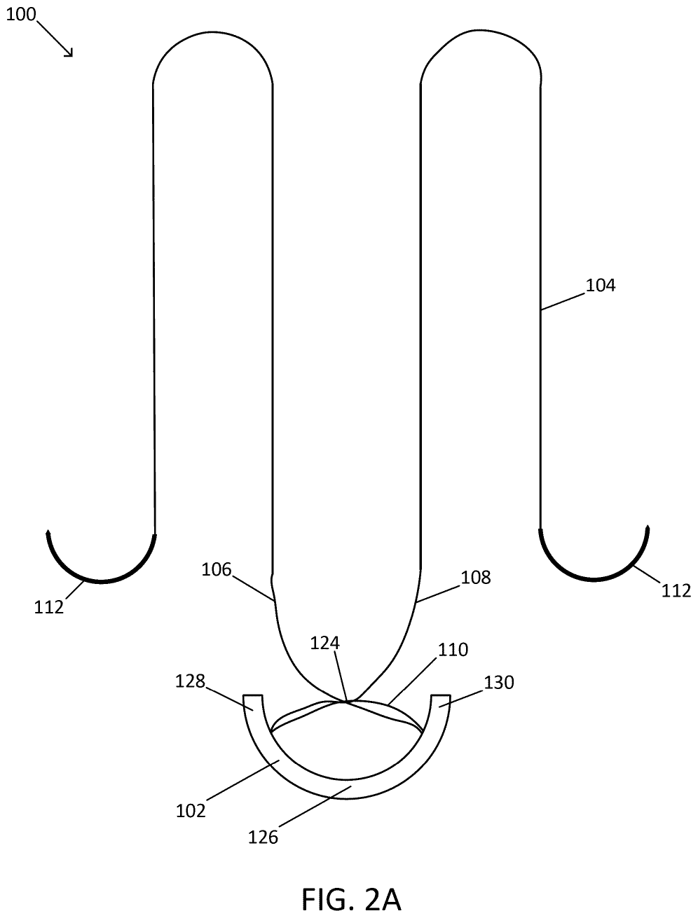

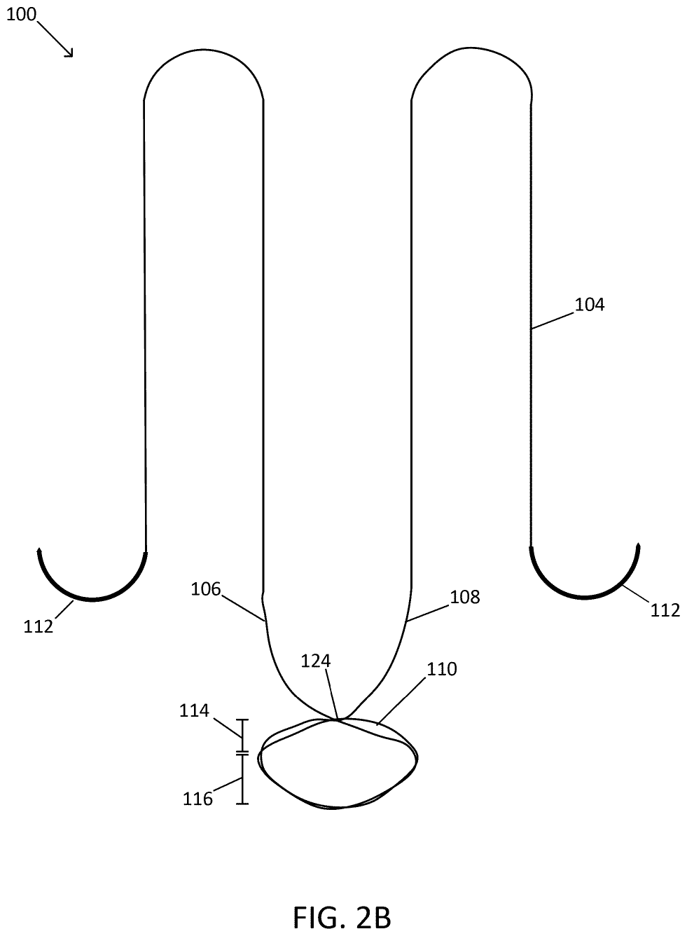

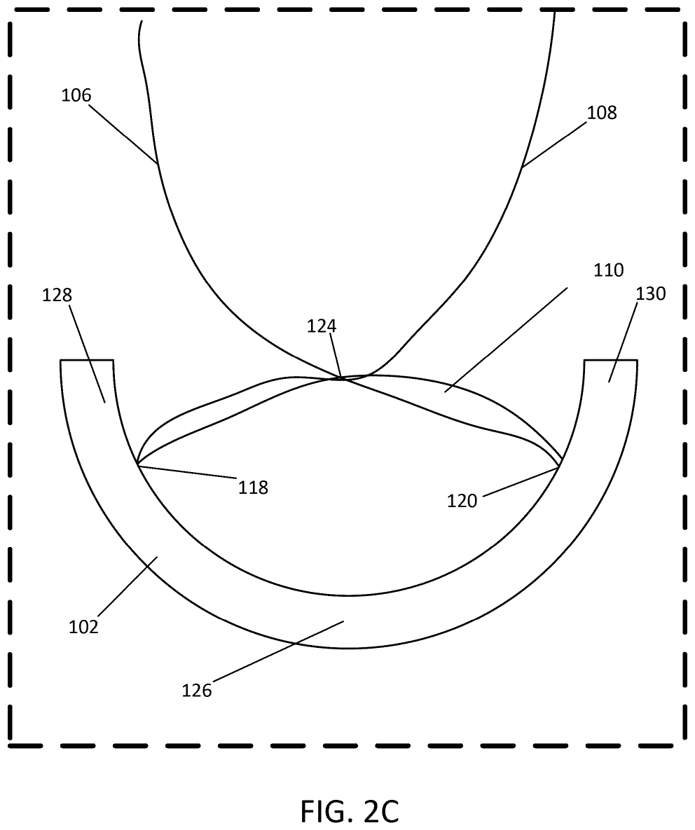

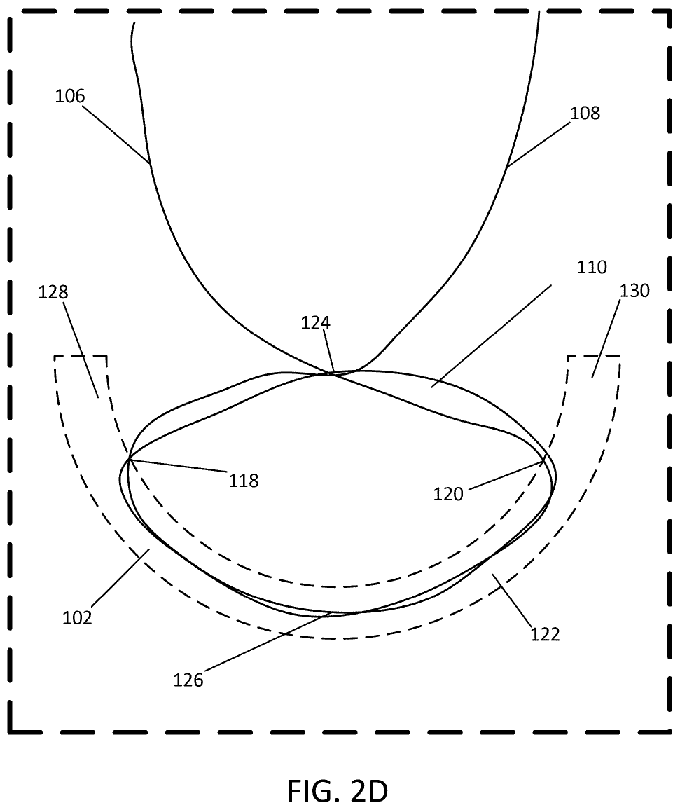

FIGS. 2a-d show an example of a surgical anchor 100 without the inserter. The surgical anchor 100 includes an anchor body 102 with a central body portion 126 and two wing portions 128, 130. The surgical anchor 100 also includes a flexible elongated member 104, with the flexible elongated member 104 including a looped section 110 and two tail sections 106, 108 extending from the looped section 110. Needles 112 are affixed to the ends of the tail sections 106, 108.

The looped section 110 of the flexible elongated member 104 includes a proximal portion 114 and a distal portion 116 (see FIG. 2b). In the example shown in FIGS. 2a-d, the distal portion 116 of the looped section 110 engages the anchor body 102 by piercing through the anchor body 102 at points 118 and 120 (see FIG. 2c), adjacent the wings 128, 130, and extending through a cavity 122 (see FIG. 2d) of the anchor body 102. As best shown in FIG. 2d, in this example, the flexible elongated member 104 loops twice through the cavity 122 of the anchor body 102. Although the flexible elongated member 104 pierces and loops through the anchor body 102 in this example, the flexible elongated member 104 is not fixed with respect to the anchor body 102, as can slide relative to the anchor body 102 when tensioned.

In other implementations, the flexible elongated member 104 may engage the anchor body 102 in other ways than what is shown in FIGS. 2a-d. For instance, in other implementations, the anchor body may be a suture tape or other deformable body that does not have a cavity, with the flexible elongated member interwoven with or otherwise engaged to the anchor body.

In the example shown in FIGS. 2a-d, the tail sections 106, 108 of the flexible elongated member 104 extend from the proximal portion 114 of the looped section 110 and are connected to one of the loops of the looped section at a proximal apex 124 of the looped section 110. More specifically, the tail sections 106, 108 pierce though one of the loops of the looped section 110 at the proximal apex 124. In other implementations, the tail sections 106, 108 may connect to the loop of the looped section 110 in other fashions, or, in still other implementations, may not pierce or otherwise connect to the proximal portion of the looped section.

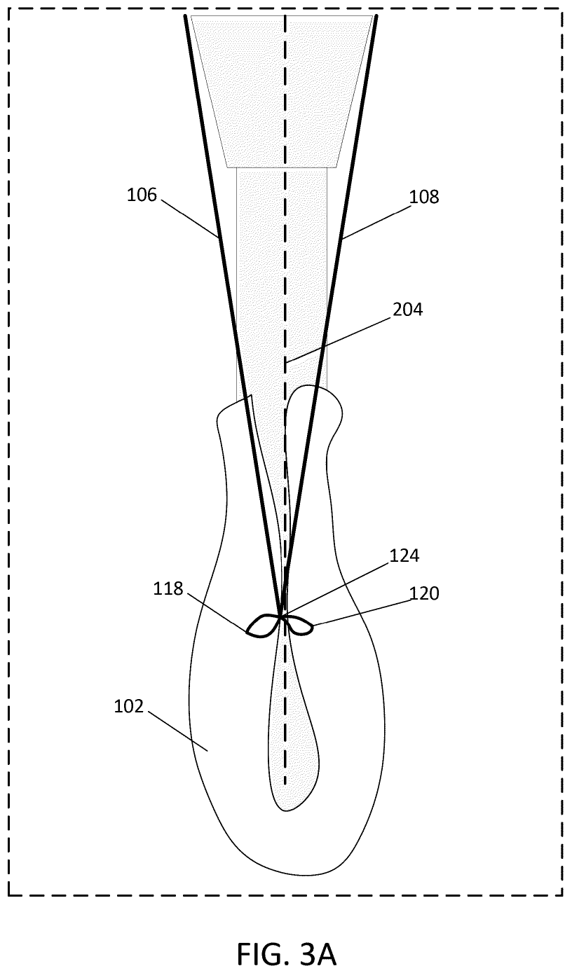

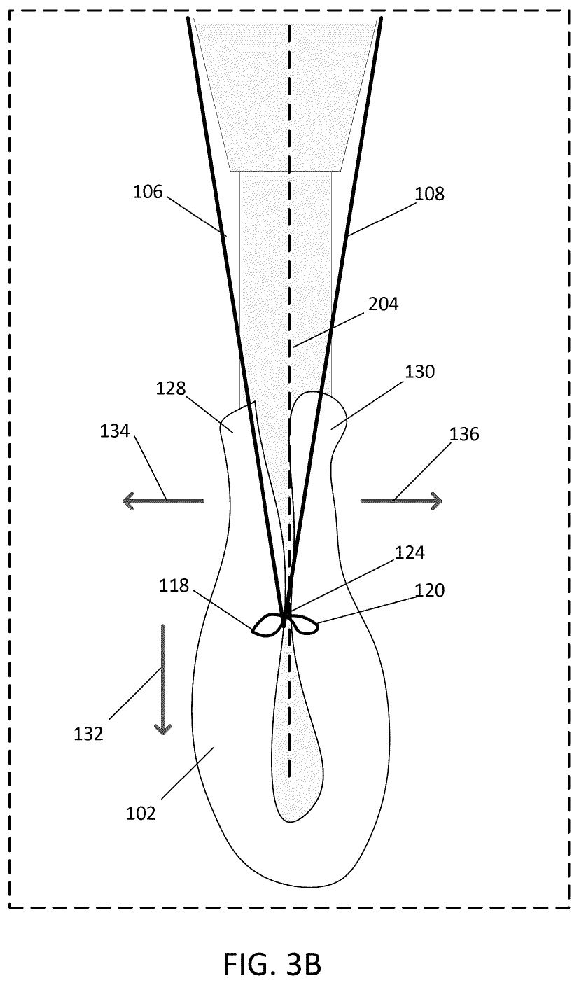

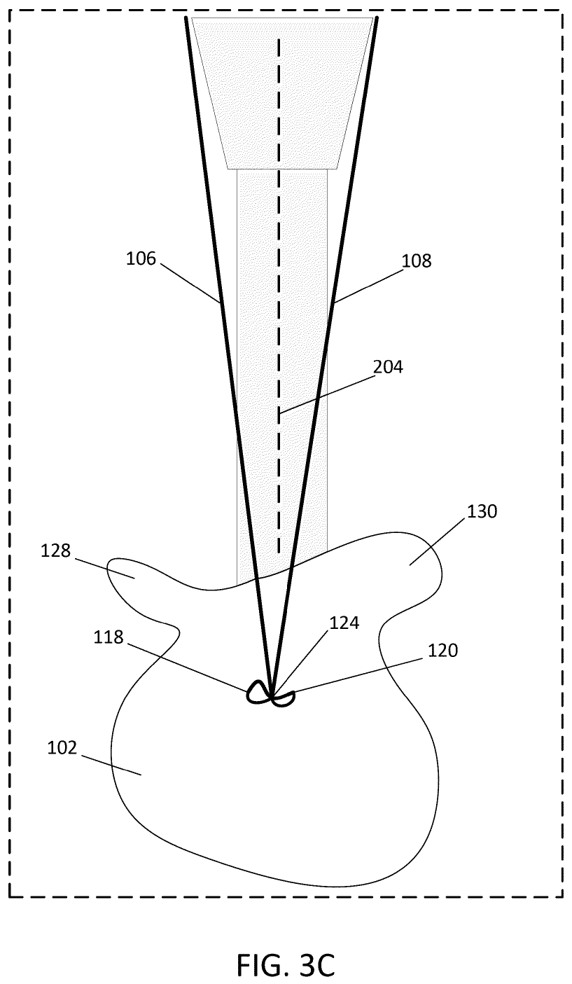

FIGS. 3a-c show the surgical anchor 100 of FIGS. 2a-d positioned on a distal tip of an inserter, illustrating deformation of the anchor body 102 from an insertion configuration (FIG. 3a) to an anchoring configuration (FIG. 3c). Pulling or otherwise tensioning tail sections 106, 108 in a proximal direction causes the looped section 110 to tighten or close. As the tail sections 106, 108 are pulled in the proximal direction, portions of the elongated member 104 will be pulled through the pierce point at the proximal apex 124 of the looped section 110 and will be pulled through the pierce points 118, 120 in the anchor body 102. In addition to causing the looped section 110 to tighten or close, this will also result in the proximal apex 124 of the looped section 110 moving distally in the direction 132 shown in FIG. 3b. The tightening or closing of the looped section 110 and distal movement of the proximal apex 124 results in the central body portion 126 of the anchor body 102 expanding laterally and the wings 128, 130 pushing out laterally, along directions 134, 136 shown in FIG. 3b.

As shown in FIGS. 3a-c, the tail sections 106, 108 of the elongated member 104 extend at non-parallel angles to the longitudinal axis 204 of the inserter. In some implementations, orienting the tail sections 106, 108 in this manner may facilitate deployment of the wings 128, 130 outwardly during deformation of the anchor body 102 by applying mechanical force in the outward direction. In some implementations, the force vector of the tail sections 106, 108 extends to the wings 128, 130 as the proximal apex 124 of the looped section is drawing towards the distal tip of the inserter. In some implementations, the tail sections 106, 108 physically press on the wings 128, 130 during deformation depending on the hole geometry of insertion.

In one implementation, the anchor body is a non-absorbable, braided polyester sleeve (size #5) that is approximately 22 mm in length, and the flexible, elongated member is a 24 inch, ultra-high molecular weight polyester suture (size 0)). In this implementation, the ends of the polyester sleeve may be heat sealed to prevent fraying, and the sutures may pierce the sleeve at approximately 4 mm from each end, with the looped section having a loop diameter of approximately 0.35 inches. In other implementations, other anchor bodies and other elongated members may be used, in these or other configurations, may be used. For example, without limitation, in other implementations the anchor body may be a sleeve, tape, or other deformable member formed of polyester fibers or another biocompatible material, and may have a length in the range of 20-25 mm, or in the range of 15-30 mm. In other implementations, the flexible elongate member may be formed of UHMWPE or another biocompatible material and may be of a size 0 to a size 5 suture. For example, without limitation, the suture or other flexible elongated member may pierce the anchor body between 1 and 10 mm from each end of the anchor body, and the looped section of the elongated member may be formed in a loop having a diameter of 0.01 inches to 0.5 inches.

### Anchoring Method

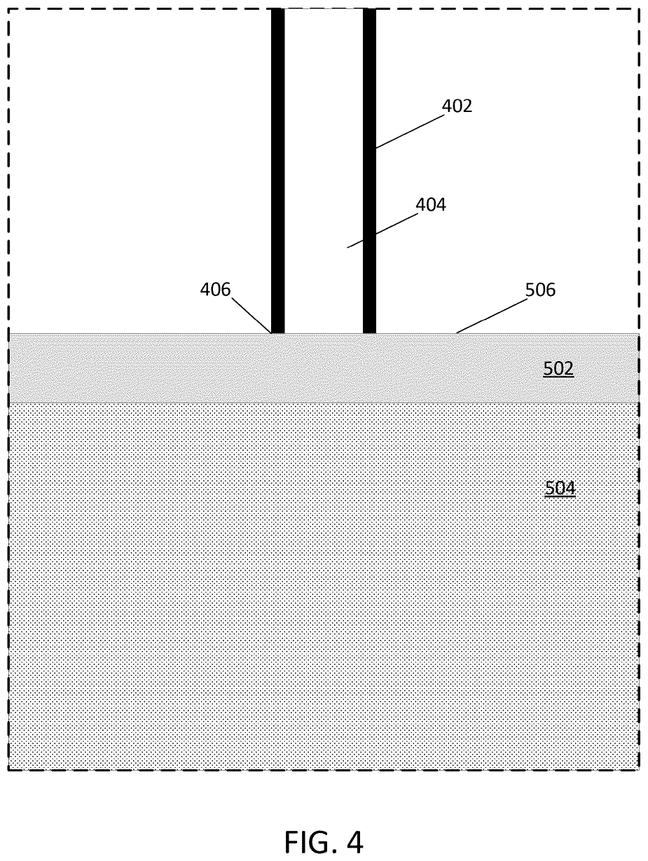

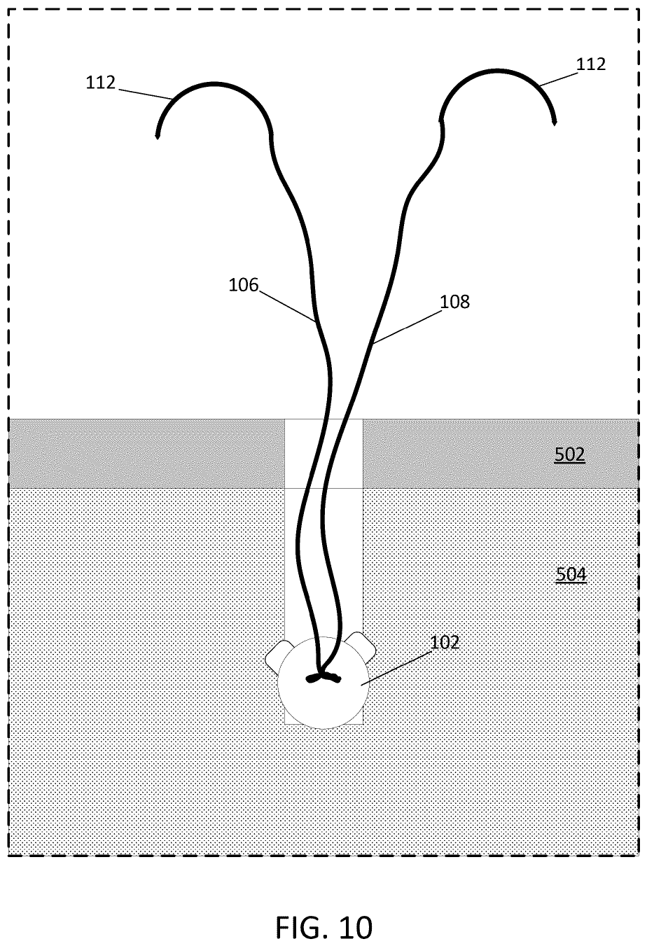

FIGS. 4-10 show one example of a surgical anchoring method using the surgical anchor 100 and inserter 200 shown in the preceding figures.

FIG. 4 shows a guide 402 initially positioned against bone. The guide 402 in this example includes an elongated opening 404 extending through it. The bone in this example includes a cortical layer 502 and a cancellous layer 504. The guide 402 is shown with its distal end 406 positioned against an outer surface 506 of the cortical layer 502.

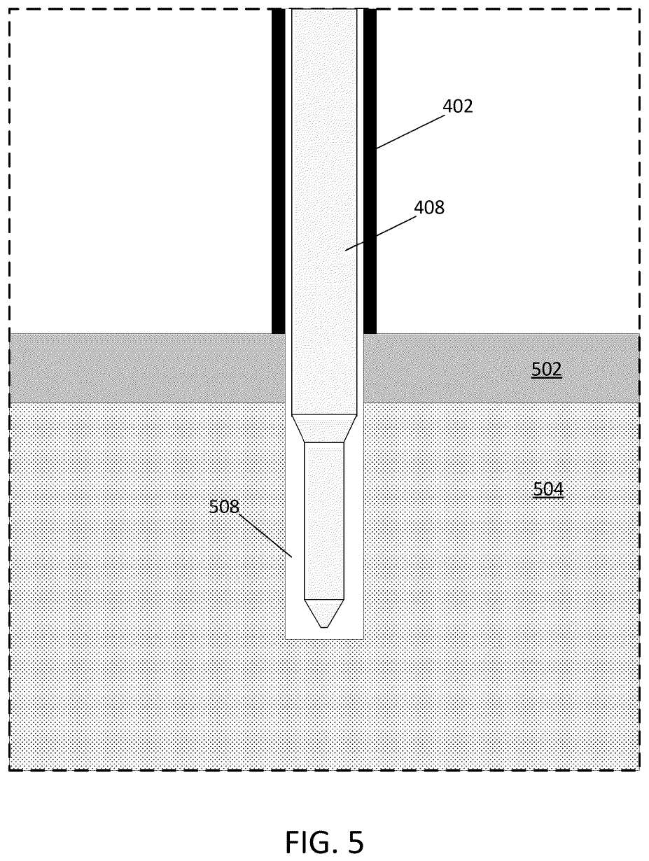

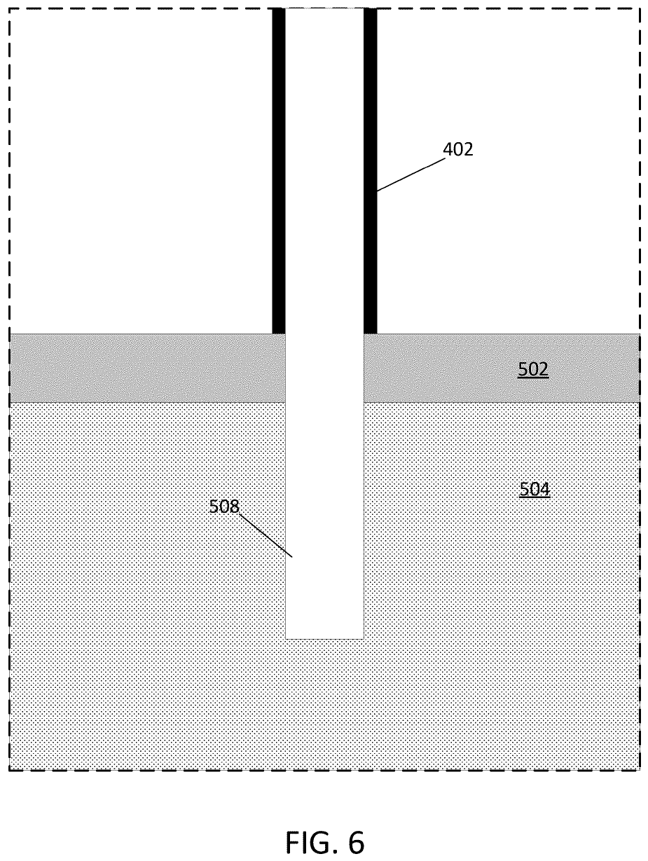

FIG. 5 shows a cutting tool 408 (in this example, a wire drill) being used to form a cavity 508 in the bone. The cutting tool 408 is inserted through the elongated opening 404 of the guide 402, and is moved distally while rotating to form the cavity 508 through the cortical and cancellous layers 502, 504. In this example, the formed cavity 508 is a blind hole. In other examples, the formed cavity may be a tunnel extending all of the way through the bone. As shown in FIG. 6, the cutting tool 408 is subsequently removed, leaving the guide 402 in place over the cavity 508. In this example, the cavity is a blind hole that is approximately 1.4 mm in diameter and 17 mm in depth. In other examples, the cavity may have a width of approximately 1.2 mm-1.6 mm, or approximately 1 mm to 2 mm, or approximately 0.5 mm to 2.5 mm. In these or other examples, the cavity may have a depth as shallow as 10 mm or shallower.

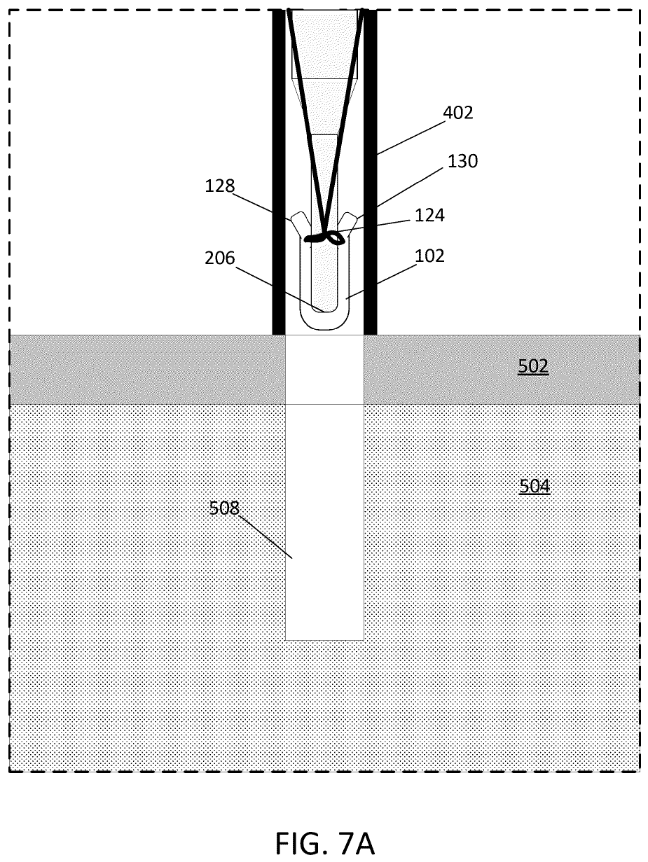

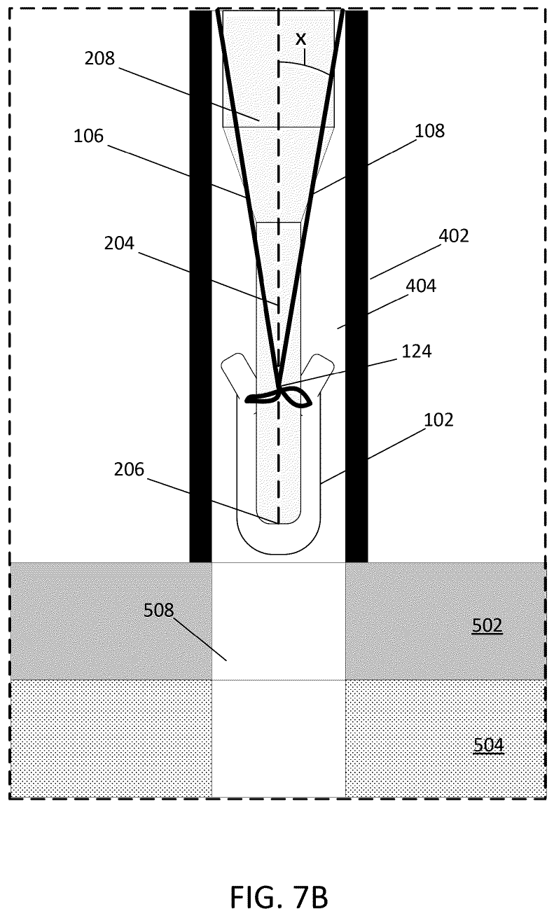

FIG. 7a shows the surgical anchor 100 subsequently passed through the elongated opening 404 in the guide 402 using inserter 200. As shown, the anchor body 102 of the surgical anchor 100 is mounted on a distal tip 206 of the inserter 200, with the anchor body 102 wrapped over the distal tip 206 such that the anchor body 102 is in a u-shape. As also shown, when the anchor body 102 is mounted on the distal tip 206 of the inserter 200, the proximal apex 124 of the looped section 110 of the flexible, elongated member 104 is distal to the proximal-most parts of anchor body wings 128, 130, and between those wings 128, 130.

As shown in FIG. 7b, when the anchor body 102 of the surgical anchor 100 is mounted on the distal tip 206 of the inserter 200, the tail sections 106, 108 extend from the proximal apex of the looped section 124 proximally through the guide's elongated opening 404, between the outer surface of the inserter's shaft 208 and the inner surface of the guide 402. As shown in FIG. 7b, when the anchor body 102 is mounted on the inserter's distal tip 206, the tail sections 106, 108 extend at extend at non-parallel angles to the longitudinal axis 204 of the inserter. In this example, the angle X is approximately 5 degrees. In other examples, the angle X may be between approximately 5 and 20. In still other examples, the angle X may be between approximately 2.5 and 25 degrees.

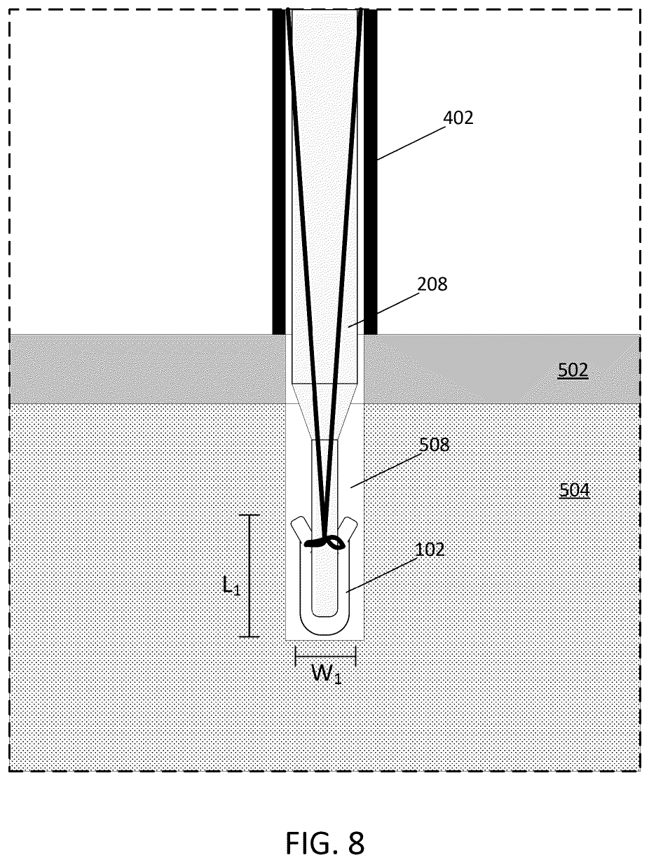

As shown in FIG. 8, the inserter 200 is used to insert surgical anchor 100 into the cavity 508. After insertion, the anchor body 102 is inserted past the cortical bone layer 502, such that the anchor body 102 is spaced apart from and below the cortical bone layer 502, in the cancellous bone layer 504. As shown in this example, the anchor body 102 is inserted until it contacts the bottom of a blind hole, although, in other examples, the anchor body 102 will not necessarily contact the bottom of the blind hole (or could be used in a cavity without a bottom, such as a bone tunnel).

In FIG. 8, the anchor body 102 is shown in its insertion configuration. In this example, when the anchor body 102 is in its insertion configuration, the anchor body 102 has a length L1 and a width W1, with the width W1 being less than the width of the cavity 508. As also shown, when the anchor body 102 is in its insertion configuration, the looped section's proximal apex 124 is between the folded-over parts of the anchor body 102, below the proximal most ends of the anchor body's wings 128, 130.

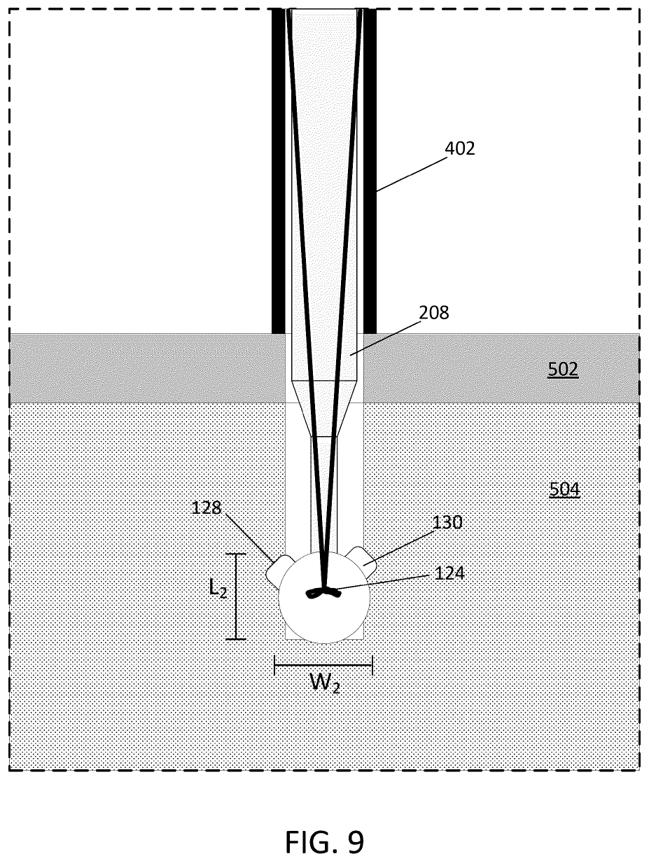

FIG. 9 shows the anchor body 102 deformed into an anchoring configuration. In this example, when the anchor body 102 is in its anchoring configuration, the anchor body 102 has a length L2 and a width W2, with the length L2 being shorter than the length L1 and the width W2 being wider than the width W1. As shown, the width W2 may be wider than the width of the cavity 508 as originally drilled, reflecting the anchor body 102 pushing into the cancellous bone 504, although, in other instances, the width W2 of the anchor body 102 in the anchoring configuration may be approximately the same as the width of the cavity 508. As also shown in FIG. 9, when the anchor body 102 is in its anchoring configuration, the wings 128, 130 are pushed out laterally, into contact with the cancellous bone 504, such that the proximal ends of the wings 128, 130 are spaced more widely apart when in the anchoring configuration than the insertion configuration.

As discussed above, tensioning tail sections 106, 108 in a proximal direction causes the anchor body 102 to deform from its insertion configuration to its anchoring configuration. As can be seen in FIGS. 8 and 9, during deformation, the looped section 110 will tighten and the connection at the looped section's proximal apex 124 between the tail sections 106, 108 and the looped section 110 will move distally, towards the distal end of the anchor body 102 and the distal tip of the inserter 206. As can also be seen in FIGS. 8 and 9, during deformation, the anchor body 102 remains spaced below the cortical bone layer 502, and does not move proximally in the bone cavity 508. As can also be seen in FIGS. 8 and 9, the shaft 208 of the inserter 200 remains stationary during deformation.

After deformation of the anchor body 102 into its anchoring configuration, the inserter 200 and guide 402 may be removed, leaving the anchor 100 firmly anchored in the patient's bone, with tail sections 106, 108 extending up and out of the bone cavity 508. Tail sections 106, 108 may be used to secure a wide variety of tissues and/or implants to the bone, including without limitation tendons, ligaments, soft-tissue repair implants, matrixes, or scaffolds, and/or tissues or constructs. Needles 112 on the tail sections 106, 108 may be used to sew the tail sections 106, 108 to the anchored tissue or construct.

### Inserter

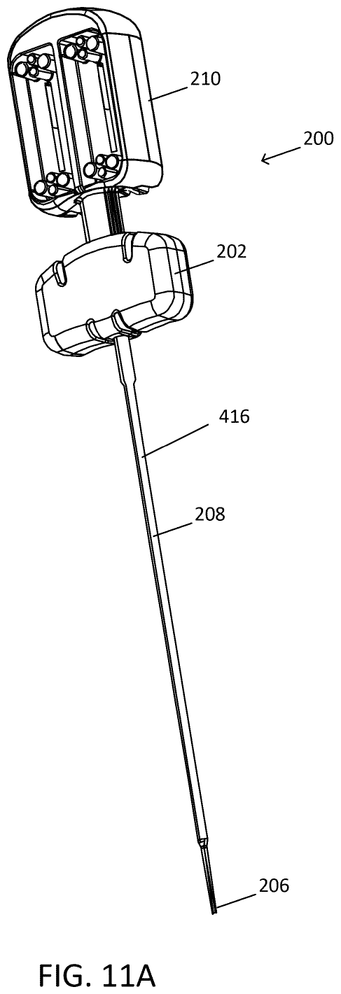

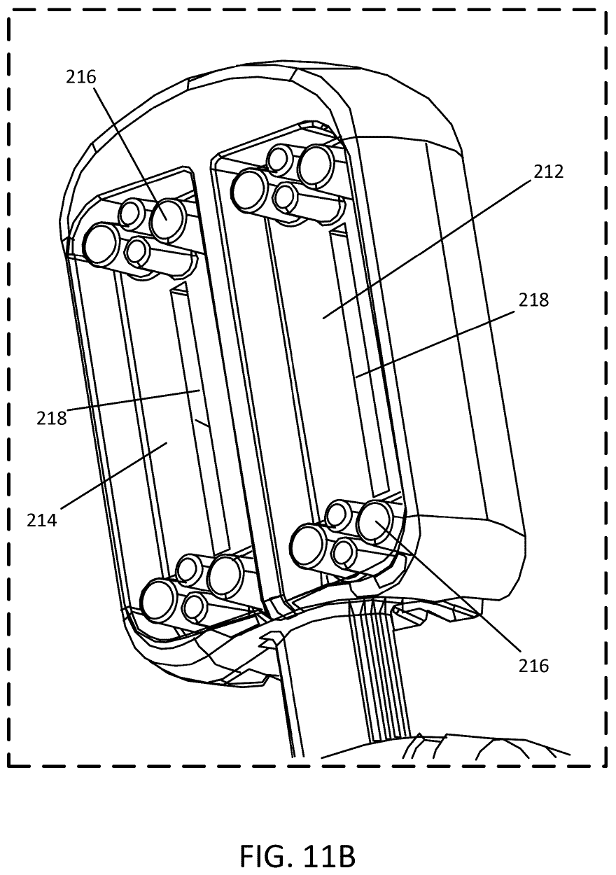

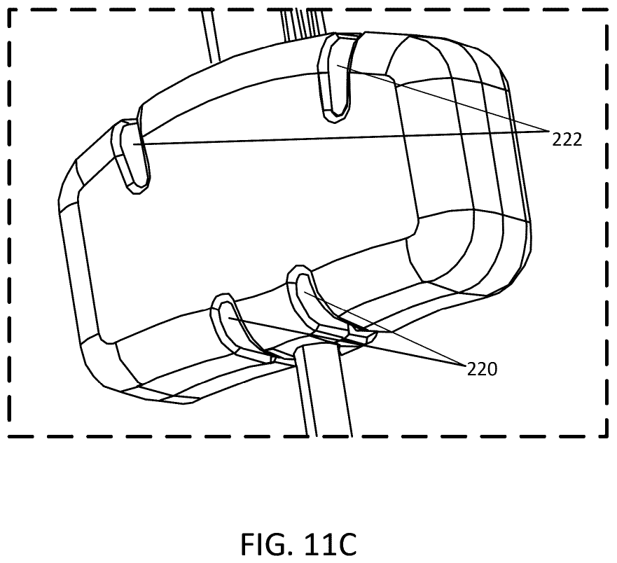

FIGS. 11-14 show examples of inserters 200 and portions thereof. The inserter 200 shown in FIG. 11A includes a inserter shaft 208 having a distal tip 206, a tensioner 202 mounted on the inserter shaft 208 in a sliding fashion, and a handle 210 at a proximal end of the inserter 200.

As shown in FIG. 11B, the handle 210 includes two recessed cavities 212, 214. Each cavity includes several posts 216, around which excess suture may be wrapped, and a slot 218, into which the needles 112 attached to the ends of suture tails may be stowed until needed. The handle 210 includes a relatively blunt proximal end, serving as a striking surface in case light malleting is required to drive the surgical anchor into the bone cavity.

As discussed previously, the inserter 200 includes a tensioner 202 that can be actuated to tension the two tail sections 106, 108 of the surgical anchor 100, causing the anchor body 102 to deform from its insertion configuration (see FIGS. 1A-B) to its anchoring configuration (see FIGS. 1C-D). In the example shown in FIG. 11C, the tensioner 202 includes distal notches 220 and proximal notches 222 around which the first and second tail sections 106, 108 of flexible, elongated member 104 may be wrapped (see, e.g. FIGS. 1A and 1C). As shown in these examples, the distal notches 220 are spaced apart from one another, which causes the tail sections 106, 108 to extend from the distal end of the inserter at non-parallel angles relative to the insertion axis. In the particular example shown in FIGS. 11A-C, the distal notches 220 are spaced apart by approximately 7 mm. In other implementations, the distal notches 220 may be spaced apart by approximately 3 to 15 mm. In still other implementations, the distal notches 220 may be spaced apart by approximately 2 to 25 mm.

In the examples shown in the figures, the tail sections 106, 108 are tensioned, and the anchor body 102 is deformed from its insertion configuration to its anchoring configuration, by squeezing the tensioner 202 in a proximal direction, towards handle 210. The inserter 200 provides visual, tactile, and audible feedback to confirm deployment of the surgical anchor 100. For visual feedback, the user can see the movement of the tensioner 202 towards the handle 210, corresponding to deployment of the surgical anchor 100. For tactile feedback, the user can feel the surgical anchor 100 deploy while squeezing the tensioner.

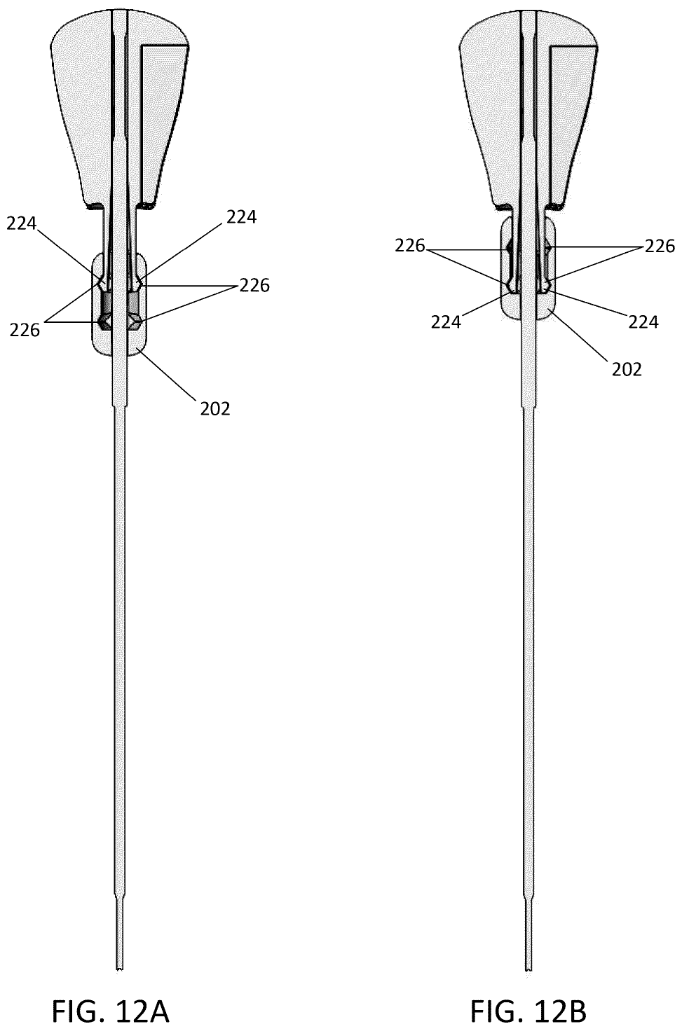

In the example illustrated in FIGS. 12A and B, the inserter includes two positive stops corresponding to the insertion (un-deployed) and anchoring (deployed) configurations of the surgical anchor 100. The positive stops are provided by the interaction of resilient arms 224 extending from the handle 210 with detents 226 in the interior of the of the tensioner 202. These positive stops will act to restrain the tensioner 202 in either the un-deployed (FIG. 12A) or deployed (FIG. 12B) positions, and will provide a tactile and audible click when the tensioner 202 is moved from the un-deployed to the deployed position.

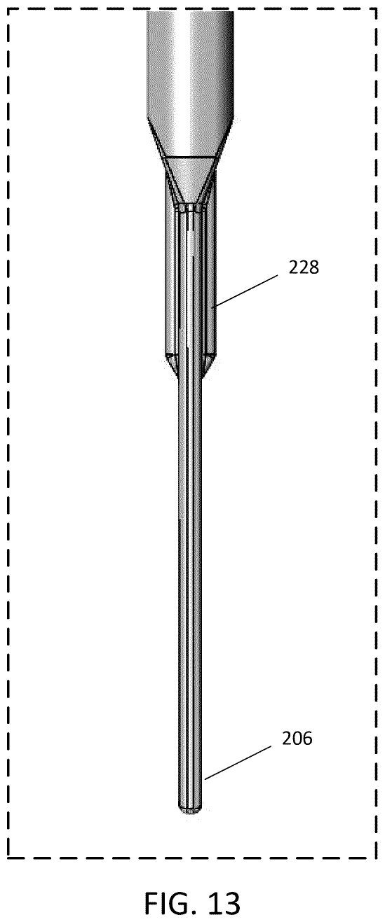

FIG. 13 shows a close up of the distal tip 206 of the inserter shaft 208. As noted earlier, in some instances it may be necessary to apply force to the inserter 200 to position the distal tip 206 of the inserter shaft 208 and the anchor body 102 mounted on it into the bone cavity 508 (e.g. as shown in FIG. 8). In the example shown in FIG. 13, the distal tip 206 includes a reinforcement 228 to mitigate against the potential of the distal tip 206 bending during insertion.

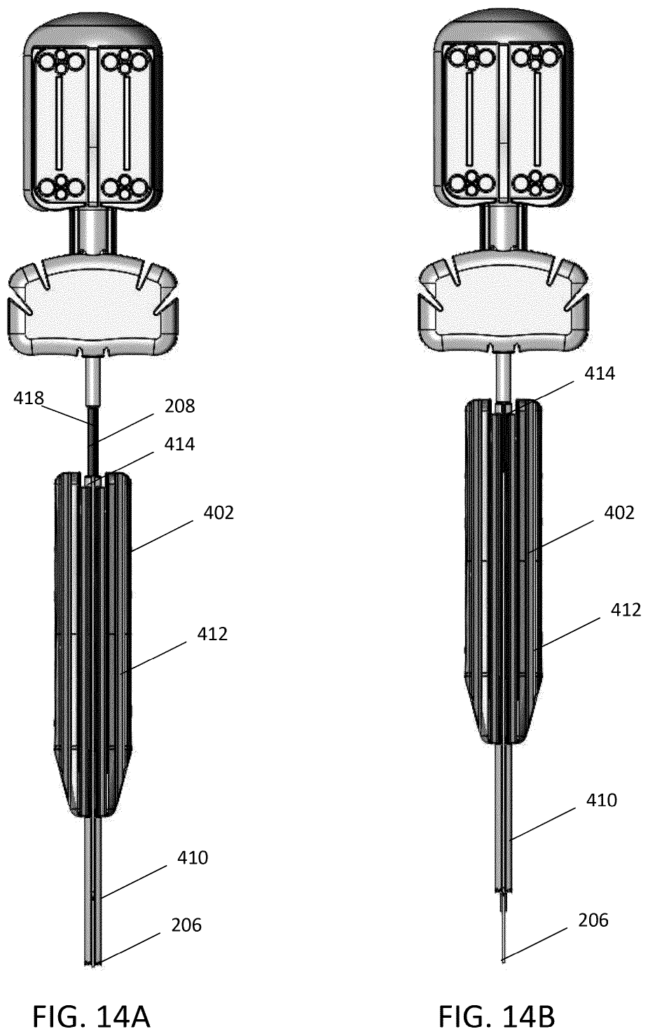

FIGS. 14A and 14B show the inserter shaft 208 positioned in a guide 402. In the example shown in these figures, guide 402 is used to both guide drilling (e.g. as shown in FIG. 5) and insertion (e.g. as shown in FIGS. 7-8). The guide 402 includes a cannulated guide 410, a handle 412, and a stop 414. FIG. 14A shows the guide 402 with the inserter shaft 208 inserted through the cannulated guide 410 to a position where the distal tip of the inserter 206 and the distal end of anchor body 102 is approximately flush with the distal end of the cannulated guide 410 (e.g. as also shown in FIG. 7). The inserter shaft 208 includes indicia (e.g. the line 416 in FIG. 11A or darkened region 418 in FIG. 14A) that visually indicates to the user when the distal tip of the inserter 206 is approximately flush with the distal end of the cannulated guide 410. FIG. 14B shows the guide 402 with the inserter shaft 208 inserted through the cannulated guide shaft 410 to a position where the inserter 200 is fully seated in the guide 402. At this point, further insertion is prevented by stop 414 contacting a shoulder on the inserter shaft 208. In the example shown in FIG. 14B, the user can confirm that the inserter 200 is fully seated because the darkened region 418 of inserter shaft 208 is fully hidden inside of the guide 402. When the inserter 200 is fully seated in the guide 402, the anchor body 102 is in a deployment position (e.g. as shown in FIG. 8).

### Testing

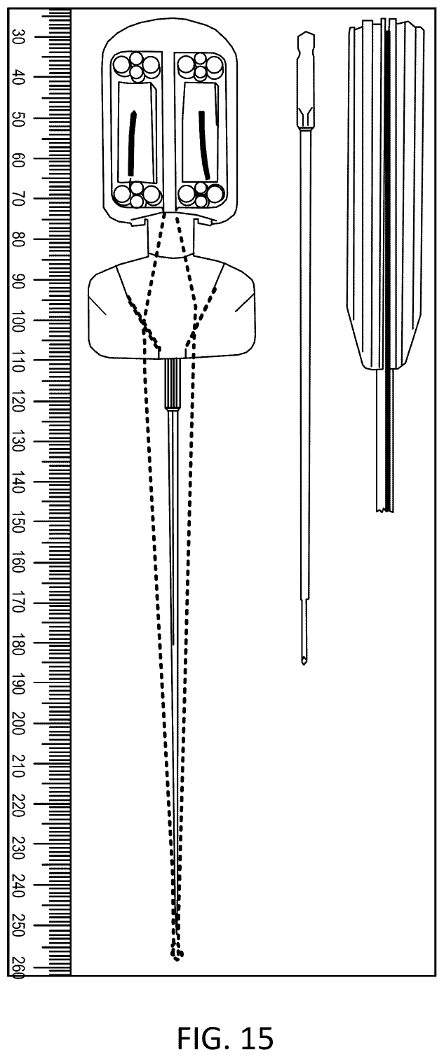

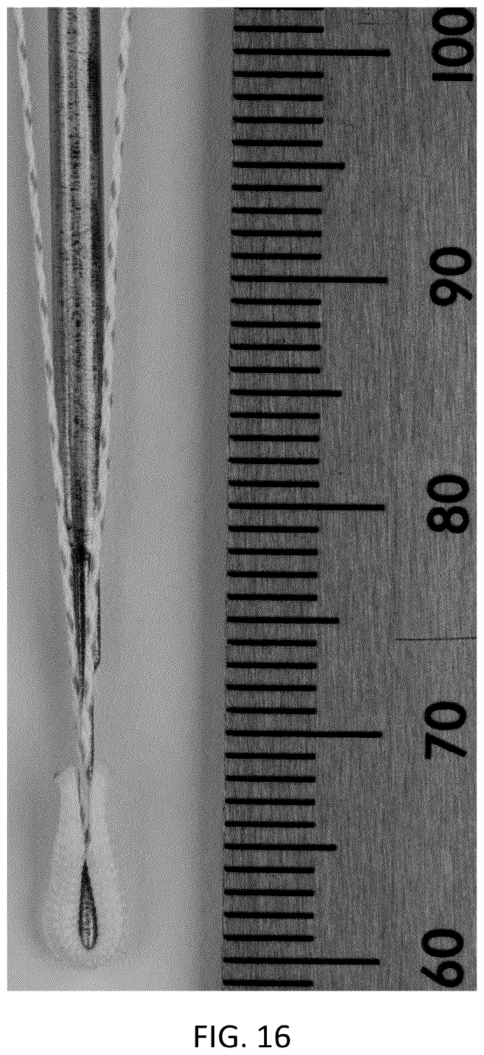

FIGS. 15-18 illustrate testing of a surgical anchoring system in accordance with this patent (referred to below as the Artelon Rivit surgical anchoring system). FIGS. 15 and 16 show the Artelon Rivit surgical anchoring system that was tested. Several samples were anchored into test blocks formed of a 3 mm thick sheet of 40 pcf polyurethane foam (simulating cortical bone) bonded with a urethane based glue over a 40 mm thick sheet of 20 pcf open cell foam (simulating cancellous bone). Pre-fatigue and post-fatigue axial pull-out tests were performed.

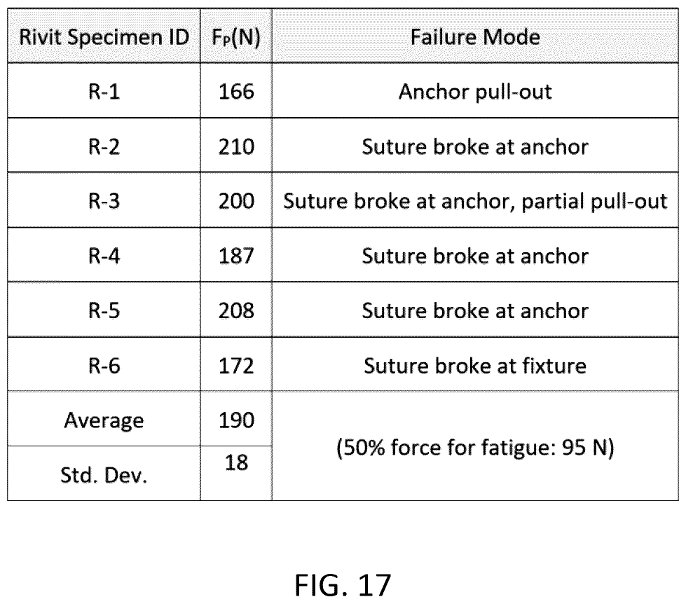

The results of the pre-fatigue pull out tests for the Artelon Rivit system are shown in FIG. 17. In the pre-fatigue pull out tests, the anchors were pre-tensioned at 10 N, with pull-out being subsequently performed in displacement control at a rate of 5 mm/min until the anchor dissociated from the foam block. The Artelon Rivit anchors had an average peak pull-out force of 190 N.

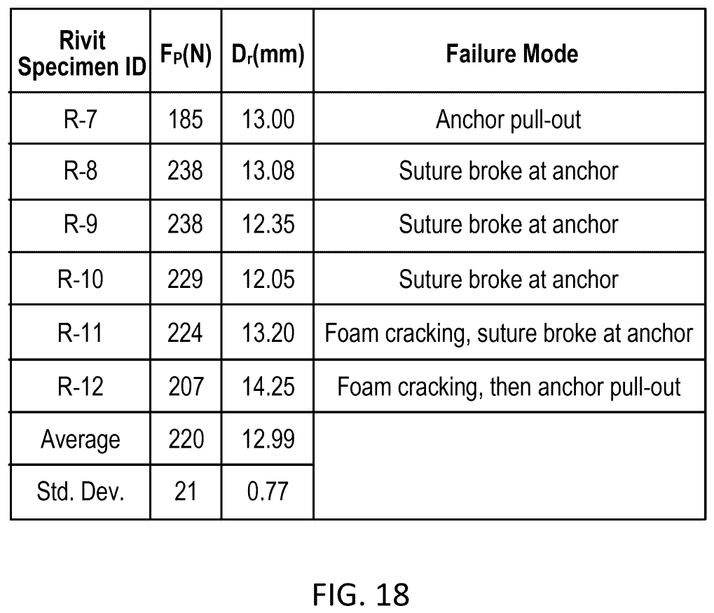

Prior to the post-fatigue test, a cyclic force was applied to each sample. A pre-tensioning load of 2 N was initially applied, followed by a loading sequence. For the loading sequence, a 10 N was applied and held for 5 seconds, followed by a cyclic force from 10-20 N being applied at 1 Hz for 10 cycles, followed by a 10 N force held for 5 seconds. Next, force was increased to the average pull-out force determined by the pre-fatigue test, and then a cyclic force from 10 N to 50% of the average static pull-out force was applied at 1 Hz for 500 cycles, after which the force was set to 10 N and held for 5 seconds. A final pull-out was performed in displacement control at a rate of 5 mm/min until the anchor dissociated from the foam block. Post-fatigue pull-out force was determined by the maximum recorded force during the test. The results of the post-fatigue pull out tests for the Artelon Rivit system are shown in FIG. 18. In the post-fatigue tests, the Artelon Rivit samples had an average peak pull-out force of 220 N and displacement during fatigue of 12.99 mm.

Additions, deletions, substitutions, and other modifications can be made to the surgical anchors and surgical anchoring systems and methods described above without departing from the scope or spirit of the inventions set out in the following claims.

What is claimed is:

1. A surgical anchor, comprising:
(a) an anchor body, the anchor body deformable from an insertion configuration to an anchoring configuration; and
(b) a flexible, elongated member, the flexible, elongated member comprising:
(i) a looped section, the looped section including a proximal portion and a distal portion, wherein at least part of the distal portion is engaged with the deformable anchor body; and
(ii) first and second tail sections, the first and second tail sections extending from a proximal portion of the looped section;
wherein the surgical anchor is configured such that tensioning the first and second tail sections causes the loop to narrow, causing the anchor body to deform to the anchoring configuration.

2. The surgical anchor of claim 1, wherein the first and second tail sections connect to the looped section at a proximal apex of the looped section.

3. The surgical anchor of claim 2, wherein the looped section is formed by two overlapping loops of the elongated member and wherein the looped section ends where the first and second tail sections connect to the looped section.

4. The surgical anchor of claim 2, wherein the looped section comprises more than one complete loop of the elongated member, wherein the elongated member, including the looped section and the two tail sections, is a single suture.

5. The surgical anchor of claim 2, wherein the surgical anchor is configured such that tensioning the first and second tail sections causes the loop to narrow and causes the connection between the looped section and the tail sections to move distally, causing the anchor body to deform to the anchoring configuration.

6. The surgical anchor of claim 1, wherein the first and second tail sections of the elongated member pierce the elongated member at the proximal portion of the looped section to connect the first and second tail sections to the looped section.

7. The surgical anchor of claim 6, wherein tensioning the first and second tail sections draw the tail sections further through the elongated member where the tail sections pierce the elongated member.

8. The surgical anchor of claim 7, wherein tensioning the first and second tail sections tightens the looped section.

9. The surgical anchor of claim 1, wherein the anchor body comprises a central body portion and two wings.

10. The surgical anchor of claim 9, wherein tensioning the first and second tail sections causes the central body to expand in size and causes the two wings to press outwardly.

11. The surgical anchor of claim 9, wherein the anchor body comprises a sleeve.

12. The surgical anchor of claim 11, wherein the looped section of the elongate member extends through a cavity of the sleeve.

13. The surgical anchor of claim 12, wherein the looped section extends out of the sleeve adjacent to the two wings.

14. The surgical anchor of claim 12, wherein the looped section of the elongate member pierces the sleeve adjacent to the two wings.

15. A surgical anchor, comprising:
(a) an anchor body, the anchor body deformable from an insertion configuration to an anchoring configuration; and
(b) a single length of suture, the single length of suture comprising:
(i) a looped section, the looped section including more than one complete loops of the single length of suture; and
(ii) first and second tail sections, the first and section tail sections extending from the looped section;
wherein the surgical anchor is configured such that tensioning the first and second tail sections causes the looped section to narrow, causing the anchor body to deform to the anchoring configuration.

16. A surgical anchoring method, the method comprising:
(a) using an inserter, inserting a surgical anchor into a bone cavity, the surgical anchor comprising:
(1) an anchor body, the anchor body deformable from an insertion configuration to an anchoring configuration; and
(2) a flexible, elongated member, the flexible, elongated member comprising:
(i) a looped section, the looped section including a proximal portion and a distal portion, wherein at least part of the distal portion is engaged with the deformable anchor body; and
(ii) first and second tail sections, the first and second tail sections connected to the looped section at the proximal portion of the looped section;
(b) tensioning the first and second tail sections to cause the connection between the tail sections and the looped section to move distally, causing the anchor body to deform to the anchoring configuration, anchoring the surgical anchor in the bone cavity.

17. The surgical anchoring method of claim 16, wherein the bone cavity extends through cortical bone and into cancellous bone, wherein inserting the surgical anchor comprises inserting the surgical anchor into the cancellous bone such that the anchor body is spaced from and below the cortical bone.

18. The surgical anchoring method of claim 17, wherein the first and second tail sections are tensioned and the anchor body deformed to the anchoring configuration while the anchor body is spaced from and below the cortical bone.

19. The surgical anchoring method of claim 17, wherein the bone cavity is between about 1 mm and 2 mm in diameter, and wherein, after anchoring the surgical anchor in the bone cavity, the surgical anchor is configured to resist a pullout force of at least 150 N.

20. The surgical anchoring method of claim 16, wherein the first and second tail sections are tensioned and the anchor body deformed to the anchoring configuration without drawing the anchor body proximally in the bone cavity.

21. The surgical anchoring method of claim 16, wherein the anchor body is on a distal end of the surgical instrument, wherein the distal end of the surgical instrument remains stationary during deformation of the anchor body.

22. The surgical anchoring method of claim 16, wherein the inserter comprises an elongated shaft extending along an insertion axis, and, wherein during tensioning of the first and second tail sections, the tail sections extend from the connection between the tail sections and the looped section at non-parallel angles relative to the insertion axis.

23. The surgical anchoring method of claim 16, wherein:
(i) the inserter comprises an elongated shaft and a tensioner;
(ii) the tensioner moveable relative to the elongated shaft between an un-tensioned position and a tensioned position;
(iii) the anchor body of the surgical anchor on a distal end of the elongated shaft; and
(iv) the first and second tail sections of the elongated member extending from the anchor body to the proximal tensioner;
wherein moving the tensioner from the un-tensioned position to the tensioned position causes the anchor body to deform to the anchoring configuration.

24. The surgical anchoring method of claim 23, wherein the inserter comprises positive stops at the un-tensioned and tensioned positions.
